(19) United States
(12) Patent Application Publication
(10) Pub. No.: US 2007/0238968 A1
(43) Pub. Date: Oct. 11, 2007

(54) Method and apparatus for providing low dose dual energy for PET attenuation correction

(76) Inventors: Vitaliy Rappoport, Knoxville, TN (US); David D. Faul, Knoxville, TN (US); Vladimir Panin, Knoxville, TN (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/784,030

(22) Filed: Apr. 5, 2007

Related U.S. Application Data
(60) Provisional application No. 60/789,341, filed on Apr. 5, 2006.

(51) Int. Cl. A61B 5/05

(52) U.S. Cl. 600/407; 600/420

(57) ABSTRACT

The apparatus and method include performing a medical image scan, including providing a contrast agent, performing a first scan at a first energy level, and performing a second scan at a second energy level, wherein radiation exposure to a patient of the first scan and the second is comparable to a single scan at a standard energy level.

### PRIORITY CLAIM TO OTHER APPLICATIONS

This application claims priority from U.S. Provisional Patent Application Ser. No. 60/789,341 filed on Apr. 5, 2006, the disclosure of which is incorporated by reference in its entirety herein.

### BACKGROUND OF THE INVENTION

1. Field of the Invention

This invention generally pertains to an apparatus and method for performing medical image scanning. More particularly, the present invention is directed to an apparatus and method of providing low dose dual energy for positron emission tomography (PET) attenuation corrections in PET/computed tomography (CT) studies.

2. Description of the Related Art

Medical imaging falls into two distinct modalities or types. Transmission imaging refers to imaging such as X-ray imaging where the imaging source (e.g., X-ray) is external to the subject and is transmitted through the subject to a detector. Emission imaging refers to imaging where the imaging source (e.g., gamma-emitting radiopharmaceutical) is internal to the subject (as a result of injection or ingestion) and is emitted from the subject to a detector. Attenuation of source radiation occurs when the source radiation passes through the subject tissue, as a result of the subject absorbing or scattering some of the radiation photons. In general it is a simple matter to determine the attenuation of a discrete transmission source, since the amount of the external source being transmitted through the subject is known, and can be compared with the amount of radiation exiting the subject. However, measurement of attenuation in emission imaging is more difficult, because the accurate amount of emission source radiation being generated in the subject that results in a quantity of radiation being detected outside the subject cannot be measured directly.

Appropriate corrections for scatter and attenuation correction are prerequisites for quantitative nuclear medicine. X-ray CT image volumes can be used to derive Linear Attenuation Coefficient (LAC) maps (“mu-maps” or “μ-maps”), suitable for compensating for attenuation in single-photon-emission-computed-tomography (SPECT) and positron-emission-tomography (PET).

In general, a transmission scan is performed at an energy level other than the energy of the emission scan. Thus, the resulting attenuation, map needs to be scaled to the actual emission energy of the scan, before it can be used to correct for attenuation in the emission reconstruction process. For source-based derived mu-maps, the conversion is simple because the discrete transmission and emission energies are known. For x-ray CT however, the transmission spectrum is continuous (and not discrete as it is the case for source-based methods of mu-map derivation), and, more importantly, depends upon the particular CT scanner and the attenuating body.

Attenuation coefficients for different types of tissue depend on the energy of the photons, and can be grouped in essentially four groups, depending on their atomic number, Z: Air, soft tissue, bone, and iron, with iron representing a class of “Very High-Z” implants, such as surgical screws, hip-replacements, or other possible very high-Z materials in the body.

X-ray CT images are calibrated so that each voxel is measured in units of Hounsfield, usually defined as:

HU^{Material}=(μ_{T}^{Material}−μ_{T}^{Water})/μ_{T}^{Water}−μ_{T}^{Air})*1000 (1),

### In this definition HU^{Water}=0 and HU^{Air}=−1000. Other definitions set HU^{Vacuum}=−1000. All clinically used CT scanners have to be calibrated to yield HU^{Water}=0 for water for all scan techniques.

In this definition μ_{T}^{Material} is the linear attenuation coefficient of a given material at an “effective” transmission energy T, and μ_{T}^{Water} is the linear attenuation coefficient of water at the same “effective” transmission energy T. The linear attenuation coefficients are “narrow beam” values, which are derived from primary photon counts only, and thus do not include any scattered photons.

Because a CT scanner emits a continuous spectrum of x-rays, an “effective” transmission energy is not easily obtained, and usually involves actual measurement of the scan object penetrating radition spectrum. The HU values are normalized for all scanners and protocols if the CT scanner for clinical practice has been properly set-up and calibrated, so that water always corresponds to HU=0 and air corresponds to HU=−1000 (it is noted that some definitions multiply by a factor of 2¹⁰=1024; such cases are included within the scope of the invention). All clinical CT scanners have to be calibrated using a vendor specific protocol to conform to this definition. However, there is no definition for densities greater than water. For instance, the same bone tissue may have different HU values when acquired with different CT scanners. The HU value of a bone specimen may even change depending on the surrounding amount of soft tissue and reconstruction parameters on the same CT scanner. Converting bone tissue accurately and adaptively to the patient is important because otherwise it may contribute largely to attenuation of emission energy.

Recently, PET/CT studies have become important clinical tools because they provide both functional (PET) and anatomical information in one study. In addition to providing diagnostic information, CT scans are used to create attenuation coefficients for PET images. The transformation of Hounsfield Units (HU) from the CT image into an attenuation correction at 511 keV is not very straightforward because of differences in energies and spectra: CT X-rays have a continuous spectrum and depend on maximum voltage (kilovolts positive, kVp) applied to the X-ray cathode. In the current implementation, the transformation consists of two kVp-dependent lines: one between air (HU=−1000) and water (HU=0), that can be described by mu=(HU+1000)*A and is universal for all energies, and another between water and bone with mu=(HU+1000)*a(kVp)+b(kVp). The intersection between these two lines is defined by a(kVp) and b(kVp). These lines were calibrated with the aid of a GAMMEX™ phantom that includes materials with electronic densities similar to human tissues, lung, fat, heart, blood, breast, and bones with different densities, and that was shown to be universal for all CT scanners.

This approach has an accuracy of a few percent in a wide range between air and dense bone (HU=>2000). However, it doesn't work for the contrast agents that are frequently used for diagnostic CT studies. Contrast agents (some concentration of Ba or I in water) look like bone for CT studies, but at 511 keV it has an attenuation value only slightly higher than that of water. For this reason, CT-based attenuation correction results in overcorrection of attenuation for PET studies when a contrast agent is present.

The magnitude of the error depends on the type of contrast, its dilution, the method of contrast administration (oral, venous, arterial, or anal), the size of the organ in which the contrast is distributed, and the circulation of the patient. Following the venous administration of 150 ml of undiluted contrast, the concentration by volume in vascularized tissues is about 0.24%. Contrast at such a low concentration is expected to significantly change the PET attenuation correction only if it is distributed in a large organ such as the liver. On the other hand, a less diluted contrast agent in the gastrointestinal tract can pool and can change the PET attenuation correction locally.

Thus, there is a need for a system and method where CT based attenuation correction can be performed without over compensation when a contrast agent is present.

### SUMMARY OF THE INVENTION

It is therefore an object of the present invention to provide an apparatus and method where Computed tomography based attenuation correction can be performed without over compensation when a contrast agent is present in accordance with an embodiment of the present invention.

It is therefore an object of the present invention to provide an apparatus and method where a contrast agent can be distinguished from bone and tissue in accordance with an embodiment of the present invention.

It is therefore an object of the present invention to provide an apparatus and method where a dual scan can be performed in which a contrast agent can be distinguished from bone and tissue in accordance with an embodiment of the present invention.

It is therefore an object of the present invention to provide an apparatus and method where the dual scan can be performed within the radiation exposure of a standard single scan.

The apparatus and method include performing a medical image scan, including providing a contrast agent, performing a first scan at a first energy level, and performing a second scan at a second energy level, wherein radiation exposure to a patient of the first scan and the second is comparable to a single scan at a standard energy level.

### BRIEF DESCRIPTION OF THE DRAWINGS

Several exemplary embodiments of the present invention will now be described in detail with reference to the accompanying drawings:

FIG. 1 is a diagram illustrating a positron emission tomography/computed tomography (PET/CT) system in accordance with an embodiment of the present invention;

FIG. 2 is a graph illustrating measurement results for a kVp-dependent, HU-to-511 keV transformation in accordance with an embodiment of the present invention;

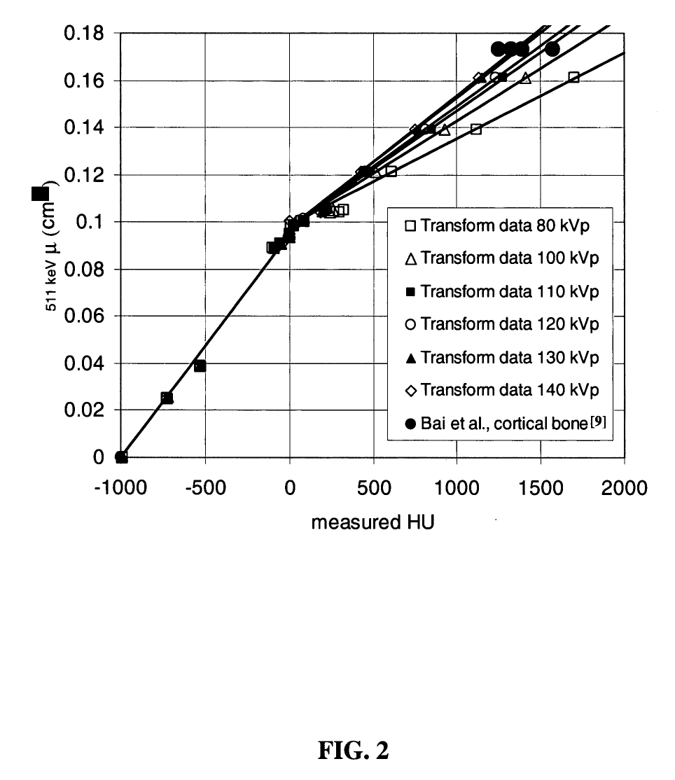

FIG. 3 is a graph illustrating a ratio of mean ROI values plus 1000HU for 80 kVp to those for 130 kVp versus HU for 80 kVp in accordance with an embodiment of the present invention;

FIG. 4 is a diagram illustrating a diagram showing trans-axial CT images in the abdominal-pelvic region for a patient scanned at both 80 kVp (left) and 140 kVp (right) in a single PET/CT scan session in accordance with an embodiment of the present invention;

FIG. 5 is a graph illustrating a plot of the ratios of mean HUs in the ROIs shown in FIG. 4 at 80 kVp, to those at 140 kVp as a function of the measured HU at 80 kVp in accordance with an embodiment of the present invention;

FIG. 6 is a graph illustrating a comparison of the 511 keV linear attenuation values obtained in accordance with an embodiment of the present invention; and

FIG. 7 is a diagram illustrating a phantom used in the study in accordance with an embodiment of the present invention.

In the drawings, the same or similar elements are denoted by the same reference numerals even though they are depicted in different drawings.

### DETAILED DESCRIPTION OF EXEMPLARY EMBODIMENTS

In the following description, a detailed description of known functions and configurations incorporated herein has been omitted for conciseness.

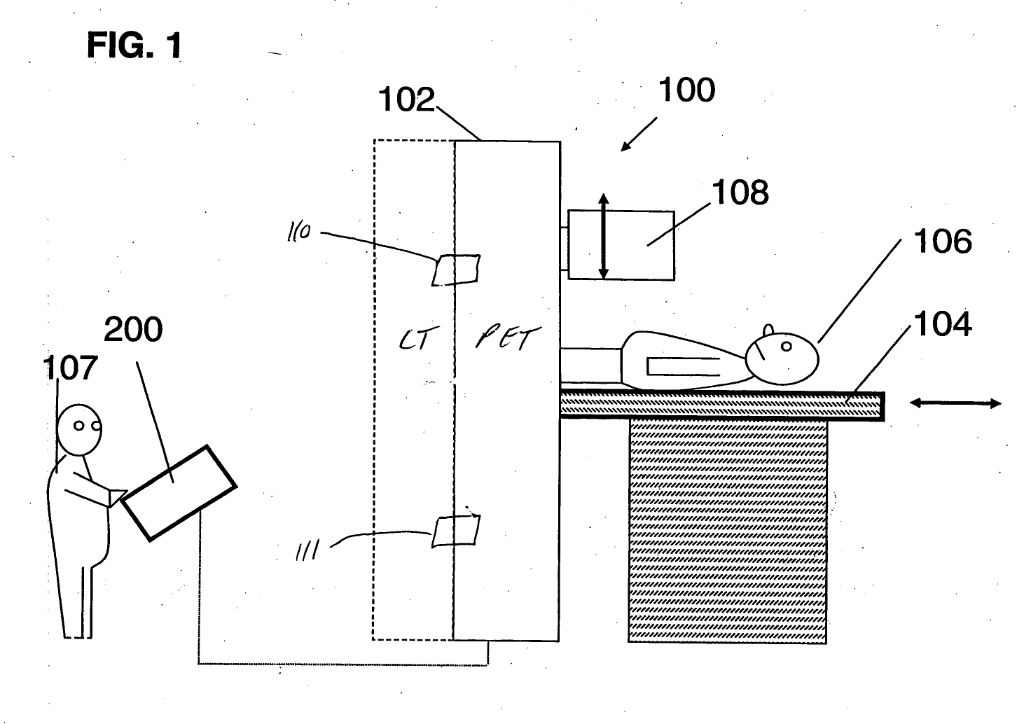

FIG. 1 is a diagram illustrating a positron emission tomography/computed tomography, (PET/CT) system in accordance with an embodiment of the present invention. Specifically, FIG. 1 depicts components of a nuclear medical imaging system 100 (i.e., having a gamma camera or a scintillation camera) which includes a gantry 102 supporting one or more detectors 108 enclosed within a metal housing and movably supported proximate a patient 106 located on a patient support (e.g., pallet) 104. Typically, the positions of the detectors 108 can be changed to a variety of orientations to obtain images of a patient's body from various directions.

The medical imaging system further comprises a processor 110 and a memory 111. The processor can run an algorithm for performing a dual scan, and the memory 111 can store the algorithm.

In many instances, a data acquisition console 200 (e.g., with a user interface and/or display) is located proximate a patient during use for a technologist 107 to manipulate during data acquisition. In addition to the data acquisition console 200, images are often developed via a processing computer system which is operated at another image processing computer console including, e.g., an operator interface and a display, which may often be located in another room, to develop images. By way of example, the image acquisition data may, in some instances, be transmitted to the processing computer system after acquisition using the acquisition console.

In accordance with an embodiment of the present invention, a dual energy computed tomography (CT) approach is provided in order to solve the problem of distinguishing bones from different concentrations of contrast agents. If the CT is performed using two energies at the same time, e.g., 80 kVp and 140 kVp, then the ratio of HU(kVp)/HU(kVp) helps to distinguish the contrast agent from bone in accordance with an embodiment of the present invention.

In addition, in accordance with an embodiment of the present invention, the dual energy CT provides some additional anatomical and physiological information, for example, regarding fat tissue. The dual-energy approach enables transformation of the linear attenuation mu at the CT effective energy to one decomposed into the Compton and photoelectric components, and allows detailed information on the composition of the material to be obtained. The dual-energy CT scan can identify more subtle differences in composition, such as for determination of the extent of fatty liver disease.

A criticism of dual energy approaches is that dual energy increases the patient's exposure to radiation. In accordance with an embodiment of the present invention, this can be mitigated by acquiring a CT diagnostic scan with technique factors, 140 kVp or 130 kVp and 60 mA, giving a body-core exposure of approximately 7 mGy, and by acquiring the second of the dual energy CT scans with moderated technique factors, 80 kVp and 30 mA, giving a body-core exposure of approximately 1 mGy. The image resulting from the second CT scan will have much higher noise, but, since the contrast agent is spread out over a large area, the increased noise does not significantly reduce the effectiveness of the dual-energy algorithm. The overall CT body-core exposure of 8 mGy is comparable to that of a single diagnostic CT scan.

It should be appreciated by those skilled in the art that the specific numbers provided are exemplary. For example a range of 60 kVp to 160 kVp can be used without departing from the scope of the present invention. Some of the determining factors concerning what specific parameters to use may comprise radiation exposure, noise, type of contrast agent used, size of area to be scanned, and size of patient and so on.

The following comprises the material and exemplary methods used. In terms of the calibration a GAMMEX® 467 electron density CP phantom (GAMMEX RMI®, Middleton, Wis., USA) was used to define a kVp-dependent transformation from HU into 511 keV linear attenuation values.

This transformation was measured on several CT scanners. In Table 1, all measured scanners are shown. The kVp settings were those that the user interfaces of the scanners offered. It should be appreciated by those skilled in the art that other brands and types of scanners can be used without departing from the scope of the present invention.

TABLE 1
(Available kVp settings for all CT scanners considered)

| CT Model | Slices | CT Model kVp Settings | | | | | | |
|---|---|---|---|---|---|---|---|---|
| Sensation | 16 | 80 | — | 100 | — | 120 | — | 140 |
| Emotion | 6 | 80 | — | — | 110 | — | 130 | — |
| Lightspeed | 16 | 80 | — | 100 | — | 120 | — | 140 |
| CXR | 4 | — | — | 100 | — | 120 | 130 | — |
| Aquilion | 16 | 80 | — | 100 | — | 120 | — | 135 |

In accordance with an embodiment of the present invention, in order to verify the algorithm, dual-energy CT measurements were performed on a Biograph 6 PET/CT scanner (Siemens MI) at three kVp settings: 110, 130 and 80 kVp. On this CT scanner, standard parameters for a conventional diagnostic CT study were 110 kVp, 100-150 mA, depending on the part of the body that was imaged (lung/abdomen) and the size of the patient. For a small region of interest (ROI) located deep inside the body, the patient exposure with this scanner was about 8 mGy. For the dual-energy measurements, technique factors of about 60 mA at 130 kVp and about 30 mA at 80 kVp were chosen, in order not to exceed the patient exposure of the conventional study at 110 kVp and 100 mA. In order to approach the same image quality for diagnosis, the dual energy CT studies were reconstructed independently and added together.

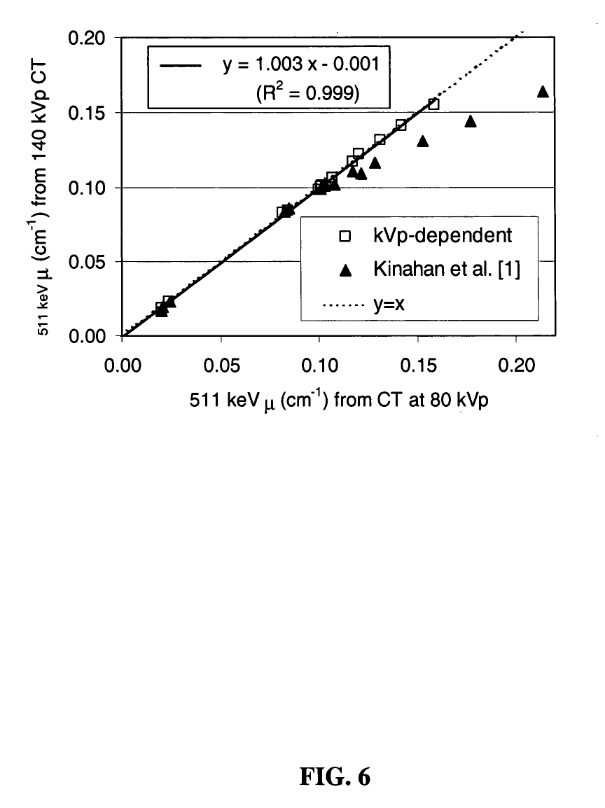

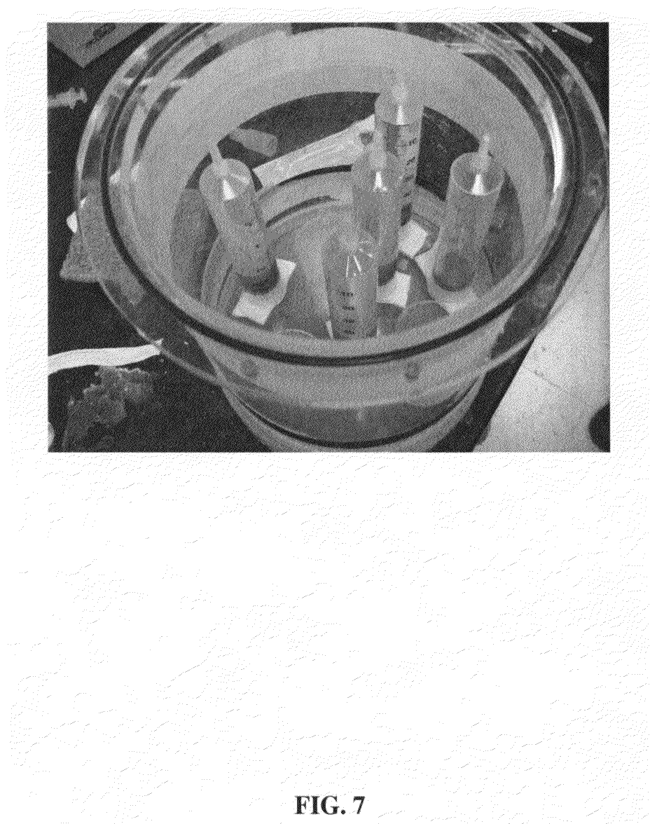

FIG. 7 is a diagram illustrating a phantom used in the study in accordance with an embodiment of the present invention. Four syringes containing contrast agent in various dilutions were placed within a cylindrical 20 cm diameter phantom, and the phantom was filled with water. The contrast agent (MD-Gastroview®, 367 mg I/ml) was diluted by volume to concentrations of 0.24%, 2%, 4%, and 6%. The syringes were filled, respectively, with each of the four solutions. See FIG. 6. It should be appreciated by those skilled in the art that other types of contrast agents can be used without departing from the scope of the present invention.

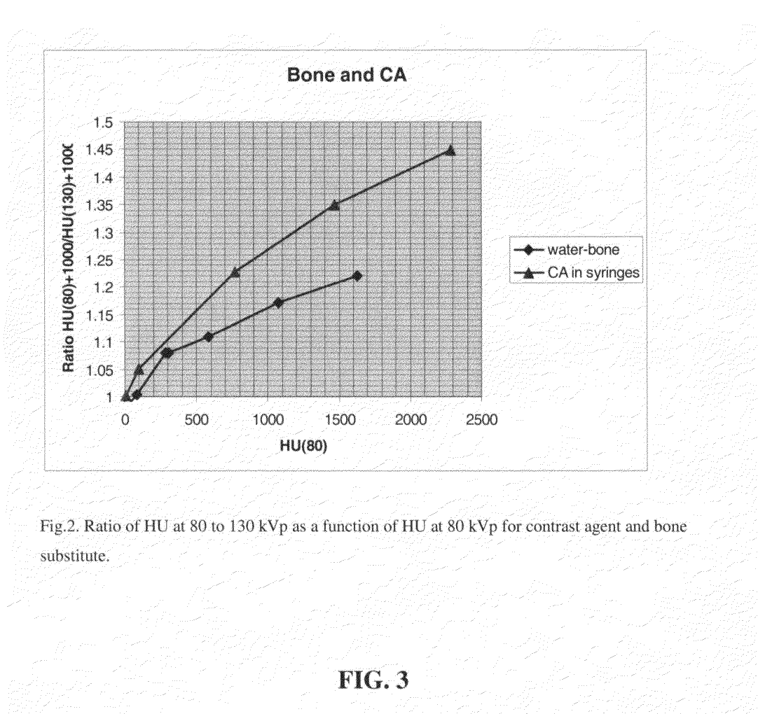

As described above, CT scans of this phantom were performed, CT images were reconstructed by the CT scanner software and hardware, and the mean H.U. values of ROIs within each syringe and within the water phantom were measured. Care was taken to maintain the ROI boundaries well within the syringe walls. For each ROI, 1000HU were added to the means, and the resulting numbers for 80 kVp were divided by those for 130 kVp. These ratios were plotted against the HU values at 80 kVp. Finally, the results from the GAMMEX® phantom were added to the plot as shown in FIG. 3. Specially, FIG. 3 shows the ratio of the mean ROI values plus 1000HU for 80 kVp to those for 130 kVp versus HU for 80 kVp. The HU values for the bone substitute and the contrast agents are higher at 80 kVp, as expected, and the HU values for the contrast agent are higher than those for the bone substitute, again as expected.

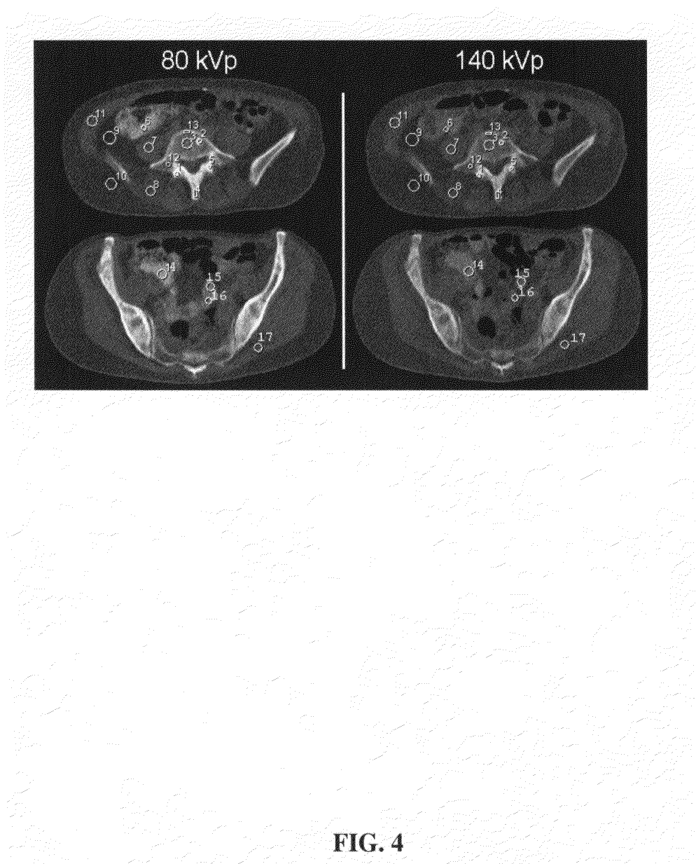

The patient 106 was scanned with a Siemens Biograph 16 scanner at 80 kVp and 140 kVp. Although not necessary for demonstrating the efficacy of this dual-energy approach, the dose of the 80 kVp scan was that of a diagnostic CT. Care was taken to limit the patient's movement between the dual-energy scans. ROIs were drawn over various tissues in the 80 kVp scan, and the mean HU for each ROI was measured with the scanner software. The ROIs were reproduced on the 140 kVp scan and the measurement was repeated as shown in FIG. 4.

FIG. 4 is a diagram illustrating a diagram showing trans-axial CT images in the abdominal-pelvic region for the same patient scanned at both 80 kVp (left) and 140 kVp (right) in a single PET/CT scan session in accordance with an embodiment of the present invention. Specifically, FIG. 4 shows ROIs for the patient study that was performed at 140 and 80 kVp. ROIs for adipose tissue (9, 10), soft tissue (7, 8, 11, 17), a range of bone tissues (1-5, 12, 13), and regions of oral contrast enhancement (6, 14-16) are defined for comparison. Care was taken to avoid potential bias due to partial volume and motion effects between the two CT scans.

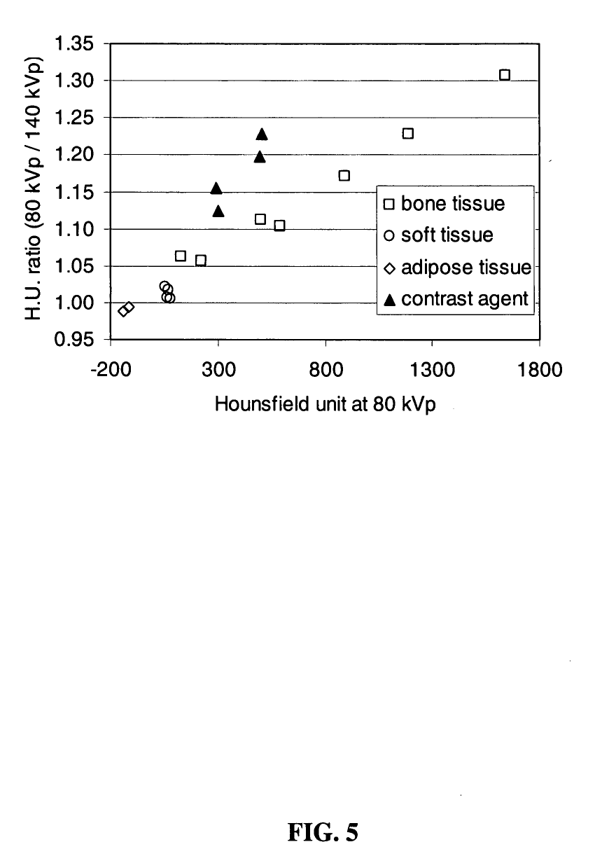

FIG. 5 is a graph illustrating a plot of the ratios of the mean HUs in the ROIs shown in FIG. 4 at 80 kVp, to those at 140 kVp as a function of the measured HU at 80 kVp in accordance with an embodiment of the present invention. Although the oral contrast agent has an HU similar to that seen in bone tissues, the ratio is higher for contrast agent than for bone at the same given HU, allowing them to be distinguished if data at more than one kVp setting are available.

Although oral-contrast-enhanced pixels in the CT image can have the same Hounsfield units as bone pixels, it is known that their associated 511 keV linear attenuation values are different. In fact, it has been shown that oral contrast in vivo, though it can result in enhancements of the CT image by hundreds of H.U., it has 511 keV linear attenuation values close to that of water. This is in contrast to bone, which has a higher linear attenuation than water at 511 keV due to its greater density. Since prior art methods and conventional kVp-dependent methods do not distinguish between oral-contrast-enhanced pixels and bone pixels in the transformation, these methods treat these pixels as bone and will overestimate the 511 keV linear attenuation for regions of oral contrast enhancement.

For routine clinical scans at 120 kVp, the resulting overestimation in reconstructed PET activity has been reported as having a maximum value in the 20-30% range, and a similar overestimation would be expected using this method, as bone and oral contrast agents are not distinguished. FIGS. 3 and 5 illustrate that with the information available from dual-energy scans, contrast agents of different concentrations (up to 10%) can be distinguished from bone, which would then allow the overestimated values to be corrected. This provides a means for the accurate treatment of contrast agents. By combining two scans, one with a reduced radiation exposure, the total radiation exposure is reduced. The overall CT image quality is the same as that of a regular scan at one kV if each kVp-dependent scan were reconstructed independently and then summed together afterwards. A further dose reduction is realizable if the correlation between the dual-energy measurements is used.

The clinical validation of the kVp-dependent transformation is clearly demonstrated in FIG. 5, which shows that anatomically equivalent regions in the CT images acquired at 80 kVp and 140 kVp are correctly transformed to the same 511 keV linear attenuation values, despite there being significant differences in the measured HU. A comparison of the 511 keV linear attenuation values obtained using the kVp-dependent transformation in patient studies from CT scans at 80 and 140 kVp, for lung, adipose, soft, and bone tissues (open boxes), is shown in FIG. 6.

FIG. 6 is a graph illustrating a comparison of the 511 keV linear attenuation values obtained in accordance with an embodiment of the present invention. Ideally, the same unique 511 keV linear attenuation values should be obtained from both measurements (i.e. y=x, dashed line). A linear regression to the data (solid line) gives y=1.003×−0.001, with an R² value of 0.999, indicating very good agreement with the ideal result. The results obtained using a prior art method show an increasing discrepancy for increasingly dense bone tissues.

Since this prior art method is optimized for 120 kVp, the results determined using this method from the CT images acquired at 80 kVp will be most in error.

While the present invention has been shown and described with reference to certain embodiments thereof, it will be understood by those skilled in the art that various changes in form and details may be made therein without departing from the spirit and scope of the invention as defined by the appended claims.

What is claimed is:

1. A method of performing a medical image scan, comprising:
providing a contrast agent;
performing a first scan at a first energy level; and
performing a second scan at a second energy level,
wherein radiation exposure to a patient of said first scan and said second is comparable to a single scan at a standard energy level.

2. The method according to claim 1, further comprising:
reconstructing an image of said first scan;
reconstructing an image of said second scan; and
combining said image of said first scan and said image of said second scan.

3. The method according to claim 2, further comprising:
correlating measurements from the first scan and measurements from the second scan, wherein said correlation results in a dose reduction.

4. The method according to claim 1, wherein a dilution of said contrast agent comprises a range from 0% to 10%.

5. The method according to claim 1, wherein said first scan and said second scan allow the contrast agent to be distinguished from bone and tissue.

6. The method according to claim 1, wherein the first scan is performed at a range of about 60 kVp to about 90 kVp.

7. The method according to claim 1, wherein the second scan is performed at a range of about 120 kVp to about 150 kVp.

8. The method according to claim 1, wherein the first scan is performed at 80 kVp and 30 ma.

9. The method according to claim 1, wherein the second scan is performed at 140 kVp and 60 ma.

10. The method according to claim 1, wherein the second scan is performed at 130 kVp and 60 ma.

11. The method according to claim 1, wherein the first scan and the second scan provide a combined radiation exposure of about 8 mGy.

12. The method according to claim 1, wherein the medical image scan is performed using computed tomography.

13. The method according to claim 1, wherein the medical image scan is performed using positron emission tomography.

14. An apparatus for providing a medical image scan comprising:
a memory adapted to store data;
a display adapted to display images; and
a processor adapted to provide a first scan at a first energy level, and a second scan at a second energy level, wherein a contrast agent is distinguished from bone and tissue.

15. The apparatus according to claim 14, wherein the processor is further adapted to reconstruct an image of said first scan, reconstruct an image of said second scan, and combine said image of said first scan and said image of said second scan.

16. The apparatus according to claim 14, wherein the processor is further adapted to correlate measurements from the first scan and measurements from the second scan, wherein said correlation results in a dose reduction.

17. The apparatus according to claim 16, wherein the first scan is performed at a range of about 60 kVp to about 90 kVp.

18. The apparatus according to claim 14, wherein the second scan is performed at a range of about 120 kVp to about 150 kVp.

19. The apparatus according to claim 14, wherein the first scan is performed at 80 kVp and 30 ma.

20. The apparatus according to claim 14, wherein the second scan is performed at 140 kVp and 60 ma.

21. The apparatus according to claim 14, wherein the second scan is performed at 130 kVp and 60 ma.

22. The apparatus according to claim 14, wherein the first scan and the second scan provide a combined radiation exposure of about 8 mGy.
